(19) United States
(12) Patent Application Publication
(10) Pub. No.: US 2008/0153920 A1
(43) Pub. Date: Jun. 26, 2008

(54) Fetal physiology during maternal surgery or diagnosis

(76) Inventors: James Dixon Reynolds, Durham, NC (US); Jonathan S. Stamler, Chaple Hill, NC (US)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 11/975,139

(22) Filed: Oct. 16, 2007

Related U.S. Application Data
(63) Continuation of application No. 10/769,912, filed on Feb. 3, 2004, now abandoned.
Application No. 10/769,912 is a continuation-in-part of application No. 10/714,980, filed on Nov. 18, 2003, now abandoned.
Application No. 10/714,980 is a division of application No. 09/919,931, filed on Aug. 2, 2001, now Pat. No. 6,676,855.
(60) Provisional application No. 60/444,868, filed on Feb. 5, 2003.

(51) Int. Cl. A61K 31/04; A61P 43/00

(52) U.S. Cl. 514/740

(57) ABSTRACT

The occurrence of hypercapneic acidosis in a fetus during a laparoscopic procedure carried out on a pregnant female, is prevented or ameliorated by inclusion in or addition to the carbon dioxide insufflation gas, of a nitric oxide donor, e.g., ethyl nitrite. Administration of nitric oxide donor in insufflation gas causes increase in fetal cerebral oxygenation.

### CROSS-REFERENCE TO RELATED APPLICATIONS

This application is a continuation of U.S. Ser. No. 10/769,912, filed Feb. 3, 2004, which is a continuation-in-part of 10/714,980, filed Nov. 18, 2003, which is a division of application U.S. Ser. No. 09/919,931, filed Aug. 2, 2001, now U.S. Pat. No. 6,676,855. U.S. Ser. No. 10/769,912 also claims the benefit of U.S. Ser. No. 60/444,868 filed Feb. 5, 2003. Each of these applications is incorporated by reference in their entirety.

### TECHNICAL FIELD

This invention is directed to negating or reducing the occurrence of hypercapneic acidosis in a fetus during a laparoscopic procedure on the fetus-carrying pregnant female and to increasing fetal cerebral oxygenation under conditions of fetal stress.

### BACKGROUND OF THE INVENTION

In a laparoscopic procedure, the insufflating gas used normally is carbon dioxide. In the case of a laparoscopic procedure on a pregnant female, the procedure can produce fetal hypercarbia and acidosis and may produce post-natal behavioral dysfunction.

Furthermore, when a fetus of a pregnant female is subjected to conditions of fetal stress, e.g., maternal pneumonperitoneum or in other cases shown by fetal monitoring, there is a need for increasing fetal cerebral oxidation.

U.S. Pat. No. 6,676,855 as indicated above is related to this patent application. U.S. Pat. No. 6,676,855 discloses that carbon dioxide pneumonperitoneum, in the case of the pregnant female, can result in impairment of blood-flow to fetus and severe hypoxemia in fetus. One embodiment disclosed in U.S. Pat. No. 6,676,855 is directed to a method for negating or reducing decrease in blood-flow in an abdominal organ which would otherwise have decreased oxygen delivery or hypercarbia or low pH because of decreased blood-flow therein because of being contacted with insufflating gas, comprising contacting said abdominal organ with a blood-flow to abdominal organ decrease preventing agent in a therapeutically effective amount. U.S. Pat. No. 6,676,855 defines “abdominal organ” as meaning an organ in the abdominal cavity or retropertoneum or a fetus or placenta.

### SUMMARY OF THE INVENTION

One embodiment of the invention herein, denoted the first embodiment, is directed to a method of preventing or ameliorating the presence of hypercapneic acidosis or hypercapnia or acidosis in a fetus of a pregnant female during a laparoscopic procedure, carried out on the pregnant female, comprising, in the course of the laparoscopic procedure, administering to said female an insufflating gas comprising carbon dioxide and an amount of a nitric oxide donor effective to prevent or ameliorate the occurrence of hypercapnia and acidosis in the fetus. This embodiment is directed to prophylaxis or treatment of a complication of a laparoscopic procedure in a pregnant female. A variation of this embodiment is directed to a method for preventing or ameliorating the presence of hypercapnia or acidosis or hypercapneic acidosis in a pregnant female comprising administering via inhalation to the pregnant female an amount of nitric oxide donor effective to prevent or ameliorate the presence of hypercapnia and acidosis in the fetus.

Another embodiment of the invention herein, denoted the second embodiment, is directed to a method for increasing fetal cerebral oxygenation under conditions of fetal stress, comprising administering to a pregnant female an insufflating gas comprising carbon dioxide and an amount of nitric oxide donor effective to increase fetal cerebral oxygenation. A variation of this embodiment is directed to a method for increasing fetal cerebral oxygenation under conditions of fetal stress, comprises administering to the pregnant female via inhalation supplemental oxygen and an amount of nitric oxide donor effective to increase fetal cerebral oxygenation and tissue blood flow.

Still another embodiment of the invention herein, denoted the third embodiment, is directed to an insufflating gas consisting essentially of carbon dioxide and from 1 to 1,000 ppm of ethyl nitrite.

The invention of the first and second embodiments involves the treatment of mammals, including humans.

The term “laparoscopic procedure” is used herein to include laparoscopic surgery as well as laparoscopic diagnosis.

Laparoscopic surgery allows surgery with minimal tissue injury and relies on a miniature video camera and surgical instruments inserted into the abdominal cavity usually through a small cut in the umbilicus. After an initial cut, a needle adapted to deliver insufflating gas is inserted. Then the insufflating gas is delivered into the abdominal cavity to expand the abdominal cavity to enable better visualization and greater workspace. The pressure resulting from the gas delivery normally should not exceed 15 mm Hg. The insufflating gas conventionally consists of carbon dioxide. After sufficient expansion is obtained, a trocar is inserted through the umbilical cut. This is used for access to insert the miniature video camera and surgical instruments. The video camera provides high resolution visualization and allows proper manipulation of the surgical instruments to carry out surgery effectively.

Diagnostic laparoscopy corresponds to laparoscopic surgery so far as initial cut and insertion of insufflating gas is concerned but the video camera is inserted to obtain diagnosis and surgery is not carried out.

Some texts available from Amazon.com in June 2001 on laparoscopy include the following: Ballantyne, G. H., Atlas of Laparoscopic Surgery; Eubanks, S. (ed), et al., Mastery of Endoscopic and Laparoscopic Surgery; Pappas, T. N., Atlas of Laparoscopic Surgery; Beshoff, J. T., et al., Atlas of Laparoscopic Retroperitoneal Surgery; MacIntyre, I. M. C., Practical Laparoscopic Surgery for General Surgeons (8/94). The whole of each of these is incorporated herein by reference.

Surgery and diagnosis can be effected on abdominal organs within the peritoneum, e.g., on liver, or on opening of the peritoneum on retroperitoneal organs, e.g., kidneys and pancreas.

The term “under conditions of fetal stress” as used herein includes maternal pneumoperitonium as well as other conditions of fetal stress, e.g., as shown by fetal monitoring.

### DETAILED DESCRIPTION

For the first and second embodiments, the nitric oxide donor is administered as part of a gas, and therefore it must normally be a gas under conditions of administration or must be converted to a gas for administration. In such case the nitric oxide donor should not have a boiling point such that the temperature required to maintain it as a gas in diluted form, i.e., admixed with carbon dioxide and other insufflating gas, if any, would harm an abdominal organ and preferably should not condense in the abdominal cavity. Nitric oxide donors that are not gases under conditions of administration are also useful, and in such case, they can be instilled into the insufflating gas from solution or inserted into the insufflating gas by other means known to those skilled in the art.

The nitric oxide donors which are administered as part of a gas are those described as blood-decrease preventing agents in U.S. application Ser. No. 09/919,931, now U.S. Pat. No. 6,676,855, the whole of which is incorporated herein by reference. These include nitric oxide donors having the formula RX—NO_{y} where R is either not present or is hydrogen/proton or C₁-C₇-alkyl and X is oxygen, sulfur, nitrogen or metal selected, for example, from the group consisting of iron, copper, ruthenium and cobalt atoms or an alkyl or alkenyl or alkylthio or alkenylthio group, containing from 1 to 7, e.g., 1 to 6, carbon atoms, which is straight chain or branched, or CF₃— or CF₃S—, and y is 1 or 2, excluding nitrous oxide. These also include nitric oxide donors which have the formula NOQ or QNO where Q is halogen, e.g., Cl, Br or F, or hydrogen, or which are NOQ or QNO generating agents, alkyl nitrososulfinates (RSO₂NO) where the alkyl group contains from 1 to 10 carbon atoms, thionitrosochloronitrite (SOClONO), thionyldinitrite (SO(ONO)₂) and thionitrites having the formula RSNO₂ where R is alkyl containing from 1 to 10 carbon atoms or is small peptide, and nitrosourea. These also include nitric oxide (NO), NO₂ and N₂O₃. A preferred nitric oxide donor for the first and second embodiments, is ethyl nitrite which is commercially available.

In the case of the first and second embodiments herein, the carbon dioxide, can be used in admixture with other insufflating gas, e.g., helium, argon or nitrogen, admixed with the carbon dioxide for administration, e.g., by conventional gas blending methods.

We turn now to the first embodiment, i.e., the method of preventing or ameliorating the presence of hypercapneic acidosis in a fetus of a pregnant female during a laparoscopic procedure, carried out on the pregnant female, comprising, in the course of the laparoscopic procedure, administering to said female an insufflating gas comprising carbon dioxide and an amount of a nitric oxide donor effective to prevent or ameliorate the occurrence of hypercapnia and acidosis in the fetus, e.g., to maintain fetal pCO₂ within 5% of baseline (i.e., before insufflation) or to reverse fetal pCO₂ or pH by an amount greater or equal to 10%.

The nitric oxide donor in the case of the first embodiment is administered in an amount effective to prevent or ameliorate the occurrence of hypercapnia and acidosis in the fetus. For this purpose, the nitric oxide donor, typically constitutes from 1 to 1,000 ppm of the gas administered, e.g., 2 to 200 ppm, e.g., 50 to 200 ppm, depending on the nitric oxide donor used, with 1 to 200 ppm usually being sufficient for ethyl nitrite, e.g., 50 to 200 ppm, but up to 1,000 ppm being used in some cases.

The nitric oxide donor can be introduced with the gas initially administered or after the start of insufflation, e.g., from 15 to 45 minutes after the start of insufflation.

As in the case of conventional laparoscopy, the pressure resulting from gas delivery should not exceed 15 mm Hg. Within this framework, the amount of gas should be sufficient to allow sufficient visualization and work space for the procedure being carried out.

We turn now to the second embodiment, i.e., to the method for increasing fetal cerebral oxygenation under conditions of fetal stress, comprising administering to a pregnant female an insufflating gas containing an amount of nitric oxide donor effective to increase fetal cerebral oxygenation. The major component of the insufflating gas can be, for example, carbon dioxide, helium, argon or nitrogen.

The method provides benefit where the fetal stress occurs because of maternal pneumoperitonium, i.e., where a laparoscopic procedure is being carried out.

The method also provides benefit where the fetal stress is because of other cause than maternal pneumoperitonium, e.g., as shown by fetal monitoring. In this case maternal pneumoperitonium is effected, i.e., an insufflating gas containing nitric oxide donor is introduced laparoscopically solely for the purpose of increasing fetal cerebral oxygenation and/or perfusion, e.g., by at least 5%.

The nitric oxide donor in the case of the second embodiment, is administered as part of the insufflating gas, i.e., in admixture with carbon dioxide and/or other insufflating gas, if any, in an amount effective to increase fetal cerebral oxygenation. The fetal cerebral oxygen level and/or general well-being of the fetus can be monitored by conventional fetal monitoring means. For the purpose of increasing fetal cerebral oxygenation, the nitric oxide donor, typically constitutes from 1 to 1,000 pm in the gas administered, e.g., 2 to 200 ppm, e.g., 50 to 200 ppm, depending on the nitric oxide door used, with 1 to 200 ppm usually being sufficient when the nitric oxide donor is ethyl nitrite, e.g. 50 to 200 ppm, but up to 1,000 ppm in some cases.

As in the case of conventional laparoscopy, the pressure resulting from gas delivery, should not exceed 15 mm Hg.

For the first and second embodiments, the gas can be delivered using a CO₂ insufflator equipped with a pressure regulator.

We turn now to the variation of the first embodiment directed to a method for preventing or ameliorating the presence of hypercapnia or acidosis or hypercapnia acidosis in a pregnant female comprising administering via inhalation to the pregnant female an amount of nitric oxide donor effective to prevent or ameliorate the presence of hypercapnia and acidosis in the fetus. The nitric oxide donor can be, for example, ethyl nitrite administered at a concentration of, for example, 1 to 100 ppm.

We turn now to the variation of the second embodiment directed to a method for increasing fetal cerebral oxygenation under conditions of fetal stress comprising administering to the pregnant female via inhalation supplemental oxygen and an amount of nitric oxide donor effective to increase fetal cerebral oxygenation and tissue blood flow. The supplemental oxygen can be, for example, 21% oxygen. The nitric oxide donor can be, for example, ethyl nitrite administered at a concentration of, for example, 1 to 100 ppm.

We turn now to the third embodiment of the invention herein, which is directed to an insufflating gas consisting essentially of carbon dioxide and from 1 to 1,000 ppm of ethyl nitrite.

The insufflating gas can contain other insufflating gas besides or in addition to carbon dioxide, e.g., helium, argon or nitrogen, but preferably contains at least 50% by volume carbon dioxide.

Preferably the insufflating gas contains from 2 to 200 ppm ethyl nitrite, very preferably from 50 to 200 ppm ethyl nitrite.

The carbon dioxide if present, other insufflating gas if any, and ethyl nitrite can be admixed to form a gas for administration by conventional gas blending methods.

The invention is illustrated by the following examples.

### EXAMPLE I

Near term pregnant sheep at gestational day 120 (term, 145 days) were surgically-instrumented with maternal and fetal catheters. After a 3+ day recovery period, ewes were anesthetized (1.5-2% isoflurane in oxygen), prepped, and then insufflated to 15 mm Hg pressure with carbon dioxide. Pneumoperitonium was maintained for at least 60 minutes after which the animals were manually deflated. For an experimental animal, 150 ppm ethyl nitrite was included in the insufflating gas 30 minutes after the start of insufflation. Maternal and fetal hydrodynamic parameters were continuously collected and then meaned at 5-minute intervals. Fetal arterial blood gases were obtained at regular intervals immediately before, during and after insufflation. Fetal pCO₂ rose from about 50 to about 80 mm Hg at 30 minutes. This was accompanied by a decrease in fetal blood pH to 7.18 and a rise in fetal arterial blood lactate concentration to 4.2 mmol/l. Introduction of ethyl nitrite in the insufflating gas was initiated 30 minutes after insufflation was started, 15 minutes after ethyl nitrite was started, pCO₂ dropped to 52, pH increased to 7.23 and arterial blood lactate concentration decreased essentially back to pre-insufflation level.

The data shows that insufflation with CO₂ was associated with significant hypercarbia and acidemia. The hypercarbia and acidemia were not controlled by active ventilation. These blood gas changes persisted in the fetus long after deflation and normalization of the ewe's physiologic status. In contrast, inclusion of ethyl nitrite in the insufflating gas, reversed the changes in fetal arterial blood gas status.

Another experiment was carried out where fetal arterial blood gas status was compared in the presence or absence of ethyl nitrite (at 150 ppm) at the start of insufflation, i.e., in one case insufflation was carried out with CO₂ with no ethyl nitrite and in the other case insufflation was carried out with carbon dioxide containing 150 ppm ethyl nitrite. Maternal pneumoperitoneum was carried out for 60 minutes. The data were averaged from three separate experiments. In the case of CO₂ alone, pCO₂ rose from about 50 to about 80 mm Hg, blood pH decreased from over 7.3 to about 7.15 and arterial blood lactate concentration rose from about 2 to about 4 mmol/l. These effects were almost completely attenuated when ethyl nitrite was in the insufflating gas.

In another experiment, insufflation with carbon dioxide alone was carried out for 70 minutes. Then 150 ppm ethyl nitrite was introduced into the insufflation gas. Fetal cerebral oxygenation was measured at regular intervals during and after insufflation. During carbon dioxide alone insufflation, oxygenated hemoglobin level varied within ±10% of baseline. Inclusion of ethyl nitrite improved fetal cerebral oxygenation to more than 30% over baseline.

Test description and data from testing is set forth in appendixes A and B of U.S. Provisional Application No. 60/444,868, the whole of which is incorporated herein by reference.

### EXAMPLE II

A 28-year old pregnant female undergoes laparoscopic surgery for gallstones. The procedure is performed with standard carbon dioxide based insufflation. Fetal monitoring during the procedure shows that the fetus develops life threatening (function degrading amount of) acidosis and hypercapnia. In particular, fetal pCO₂ increases from about 50 to about 80 mm Hg and pH drops from 7.4 to 7.1. Then ethyl nitrite at 100 ppm is added into the insufflation gas, whereupon fetal pCO₂ decreases to about 50 mm Hg and pH rises to 7.25. The ethyl nitrite administration also causes fetal cerebral oxygenation to increase to more than 20% over baseline.

### EXAMPLE III

A 28-year old pregnant female undergoes laparoscopic surgery for gallstones. The procedure is performed with carbon dioxide containing 100 ppm ethyl nitrite for the insufflation gas. Fetal pC0₂ and fetal pH are preserved.

### EXAMPLE IV

A 23-year old black female, 32 weeks pregnant, undergoes laparoscopic evaluation for right lower quadrant pain. Thirty minutes into the procedure, fetal PO₂, measured by an electrode placed on the head, is 7 mm Hg. 100 ppm ethyl nitrite is added to the carbon dioxide insufflating gas, and the fetal PO₂ increases to 25 mm Hg.

### EXAMPLE V

A 23-year old black female undergoes laparoscopic evaluation for right lower quadraul pain. The procedure is performed with carbon dioxide containing 100 ppm ethyl nitrite. Fetal P0₂ and tissue blood flow are preserved.

### EXAMPLE VI

During pregnancy, a fetus becomes distressed as indicated by a non-reassuring heart rate. The mother is administered into her lung 21% oxygen containing 10 ppm ethyl nitrite via inhalation through a face mask. The heart rate of the fetus becomes normal. A c-section delivery is not necessary.

### VARIATIONS

Variations of the above will be obvious to those skilled in the art. Therefore, the scope of the invention is defined by the claims.

What is claimed is:

1. A method of preventing or ameliorating the presence of hypercapneic acidosis or hypercapnia or acidosis in a fetus of a pregnant female during a laparoscopic procedure carried out on the pregnant female, comprising, in the course of the laparoscopic procedure, administering to said female an insufflating gas comprising carbon dioxide and an amount of a nitric oxide donor effective to prevent or ameliorate the occurrence of hypercapnia and acidosis in the fetus.

2. The method of claim 1 where the nitric oxide donor is ethyl nitrite.

3. A method for increasing fetal cerebral oxygenation under conditions of fetal stress, comprising administering to a pregnant female an insufflating gas comprising an amount of nitric oxide donor effective to increase fetal cerebral oxygenation.

4. The method of claim 3 where the nitric oxide donor is ethyl nitrite.

5. The method of claim 4 where the ethyl nitrite is present in the insufflating gas in an amount ranging from 1 to 1,000 ppm.

6. The method of claim 3 where the insufflating gas consists essentially of carbon dioxide and from 1 to 1,000 pm ethyl nitrite.

7. The method of claim 6 where the insufflating gas consists essentially of carbon dioxide and from 50 to 200 ppm ethyl nitrite.

8. A method for increasing fetal cerebral oxygenation under conditions of fetal stress comprising administering via inhalation to a female pregnant with the fetus supplemental oxygen and an amount of nitric oxide donor effective to increase fetal cerebral oxygenation.

9. The method of claim 8 where the nitric oxide donor is ethyl nitrite which is administered at a concentration of 1 to 100 ppm.

10. A method of preventing or ameliorating the presence of hypercapnia or acidosis or hypercapneic acidosis in a fetus of a pregnant female comprising administering via inhalation to the pregnant female an amount of nitric oxide donor effective to prevent or ameliorate the occurrence of hypercapnia and acidosis in the fetus.

11. The method of claim 10 where the nitric oxide donor is ethyl nitrite which is administered at a concentration of 1 to 100 ppm.
